(19) United States
(12) Patent Application Publication
(10) Pub. No.: US 2025/0051712 A1
(43) Pub. Date: Feb. 13, 2025

(54) APPARATUS FOR OBSERVING CELL COMPRISING MOVABLE IMAGE PHOTOGRAPHING DEVICE AND METHOD OF OBSERVING CELL USING THE SAME

(71) Applicant: MEDIKAN CO., LTD., Seoul (KR)

(72) Inventor: HeeYoung LEE, Seoul (KR)

(73) Assignee: MEDIKAN CO., LTD., Seoul (KR)

(21) Appl. No.: 18/724,852

(22) PCT Filed: Dec. 30, 2022
(86) PCT No.: PCT/KR2022/021708

§ 371 Date: Jun. 27, 2024

(30) Foreign Application Priority Data
Dec. 30, 2021 (KR) 10-2021-0192190

(51) Int. Cl. C12M 1/34; C12M 1/00; C12M 1/36

(52) U.S. Cl. CPC C12M 41/36; C12M 41/08; C12M 41/12; C12M 41/48

(57) ABSTRACT

Provided is an apparatus for observing cells including a movable image photographing device, which includes a light-transmitting culture vessel in which a plurality of light-transmitting attachment members are irregularly arranged, wherein a plurality of cells are attached to and cultured on the plurality of light-transmitting attachment members, an image photographing device movable within the housing and configured to photograph the plurality of cells attached to and cultured on the plurality of light-transmitting attachment members irregularly arranged in the light-transmitting culture vessel, and a light source irradiating light to the plurality of cells attached to and cultured on the plurality of light-transmitting attachment members irregularly arranged in the light-transmitting culture vessel, to allow the image photographing device to photograph each of the plurality of cells attached to and cultured on the plurality of light-transmitting attachment members irregularly arranged in the light-transmitting culture vessel, to be recognizable. Accordingly, not only an image of cells is obtained when a device for obtaining an image of cells is in a stopped state, but also an image of cells cultured in an area where an image of cells is difficult or impossible to obtain is obtained when the device for obtaining an image of cells is in a stopped state, thereby obtaining an image of cells cultured in all areas, not in a limited area, so that observation efficiency may be greatly improved.

### TECHNICAL FIELD

The present disclosure relates to an apparatus for observing cells and a method of observing cells using the same, and in particular, to an apparatus for observing cells comprising a movable image photographing device and a method of observing cells using the same.

### BACKGROUND ART

Cells are observed for various purposes. For example, during cell culture, it is necessary to observe cells to take appropriate measures for good cell culture as necessary by observing the cells being cultured.

According to the related art, there are various apparatus and methods for observing cells.

For example, Korean Patent No. 10-0813915 discloses an apparatus for obtaining an image of cells in culture without opening an incubator. Here, a cell culture observing apparatus is described, in which light emitted from lighting located in a side surface of an incubator is reflected by a reflection plate arranged above the incubator to be irradiated to cells in the incubator, an image of the cells is obtained using a camera located under the incubator, and the image of the cells are displayed on a display portion using signals output from the camera.

Korean Patent No. 10-1352639 discloses an apparatus in which information about the state of cell culture may be easily obtained by analyzing the area of a culture vessel occupied by cells during cell culture. Here, a cell culture observing apparatus is described, which comprises a culture vessel support for supporting a culture vessel accommodating cells and a media and an imaging device for obtaining an image of an area in a bottom portion of the culture vessel occupied by cells or an image of cells in a specific portion of the bottom portion of the culture vessel.

However, such apparatuses or methods for observing cells according to the related art obtain images of cells when the camera or imaging device for obtaining cell images is in a stopped state. Accordingly, as it is difficult or impossible to obtain an image of cells in culture in an area in which an image of cells is difficult or impossible to obtain, while the camera or imaging device is in a stopped state, it is a problem that only an image of cells cultured in a limited area may be obtained.

Thus, there is a need for an apparatus or method for observing cells capable of obtaining not only an image of cells when a device for obtaining the image of cells is in a stopped state, but also an image of cells cultured in an area in which obtaining an image of cells is difficult or impossible, thereby enabling obtaining an image of cells cultured in all areas, not in a limited area.

### DESCRIPTION OF EMBODIMENTS

### Technical Problem

It is an objective of the present disclosure to provide an apparatus for observing cells comprising a movable image photographing device and a method of observing cells using the same, in which not only an image of cells is obtained when a device for obtaining an image of cells is in a stopped state, but also an image of cells cultured in an area where an image of cells is difficult or impossible to obtain is obtained when the device for obtaining an image of cells is in a stopped state, thereby obtaining an image of cells cultured in all areas, not in a limited area, so that observation efficiency may be greatly improved.

It is another objective of the present disclosure to provide an apparatus for observing cells comprising a movable image photographing device and a method of observing cells using the same, in which not only an image of cells cultured in one culture vessel is obtained, but also an image of cells cultured in a plurality of culture vessels is obtained, thereby greatly improving the observation efficiency of cells cultured in a plurality of culture vessels.

It is another objective of the present disclosure to provide an apparatus for observing cells comprising a movable image photographing device and a method of observing cells using the same, in which an image of cells cultured in a culture medium in a culture vessel is easily obtained, thereby greatly improving the observation efficiency of cells cultured in a culture medium in a culture vessel.

It is another objective of the present disclosure to provide an apparatus for observing cells comprising a movable image photographing device and a method of observing cells using the same, in which a device for obtaining an image of cells is remotely driven as necessary, thereby greatly improving driving efficiency.

The objectives to be achieved by the disclosure are not limited to the above-described objectives, and other objectives that are not mentioned herein would be clearly understood by a person skilled in the art from the description of the disclosure.

### Solution to Problem

According to an embodiment of the present disclosure, an apparatus for observing cells including a movable image photographing device includes a light-transmitting culture vessel in which a plurality of light-transmitting attachment members are irregularly arranged, wherein a plurality of cells are attached to and cultured on the plurality of light-transmitting attachment members, an image photographing device movable within the housing and configured to photograph the plurality of cells attached to and cultured on the plurality of light-transmitting attachment members irregularly arranged in the light-transmitting culture vessel, and a light source irradiating light to the plurality of cells attached to and cultured on the plurality of light-transmitting attachment members irregularly arranged in the light-transmitting culture vessel, to allow the image photographing device to photograph each of the plurality of cells attached to and cultured on the plurality of light-transmitting attachment members irregularly arranged in the light-transmitting culture vessel, to be recognizable.

In the present embodiment, the culture vessel may be filled with a culture medium to culture cells to a certain height to submerge the plurality of cells. In the present embodiment, the housing may maintain the temperature within a range in which cell culture is possible. In the present embodiment, the image photographing device may be further configured to photograph, from under the culture vessel across a lower surface of the culture vessel, the plurality of cells attached to and cultured on the plurality of light-transmitting attachment members irregularly arranged in the culture vessel. In the present embodiment, while moving within the housing, the light source may irradiate light to the plurality of cells attached to and cultured on the plurality of light-transmitting attachment members irregularly arranged in the culture vessel. In the present embodiment, the light source may include a first light source irradiating light, from above the culture vessel, to the plurality of cells attached to and cultured on the plurality of light-transmitting attachment members irregularly arranged in the culture vessel. In the present embodiment, the light source may include a second light source irradiating light, from a side portion of the culture vessel to the plurality of cells attached to and cultured on the plurality of light-transmitting attachment members irregularly arranged in the culture vessel.

In the present embodiment, the apparatus may further include a reflection portion reflecting the light irradiated from the light source to the plurality of cells attached to and cultured on the plurality of light-transmitting attachment members irregularly arranged in the culture vessel. In the present embodiment, the reflection portion may be located within an irradiation angle range of the light source and may reflect the light irradiated from the light source to the plurality of cells attached to and cultured on the plurality of light-transmitting attachment members irregularly arranged in the culture vessel. In the present embodiment, the reflection portion may be located opposite the light source and may reflect the light irradiated from the light source to the plurality of cells attached to and cultured on the plurality of light-transmitting attachment members irregularly arranged in the culture vessel.

In the present embodiment, the apparatus may further include a controller configured to control the image photographing device and the light source. In the present embodiment, the controller may be connected to an external server through wired/wireless communication, and the image photographing device or the light source may be controlled by the external server. In the present embodiment, the server may receive an image of the plurality of cells photographed by the image photographing device. In the present embodiment, the server may determine a culture state of the plurality of cells by comparing information about the image of the plurality of cells photographed by the image photographing device with information about an image of the plurality of cells previously stored. In the present embodiment, the apparatus may further include a display portion displaying an image of the plurality of cells photographed by the image photographing device.

According to another embodiment of the present disclosure, a method of observing cells using an apparatus for observing cells including a movable image photographing device includes accommodating, in a housing, a light-transmitting culture vessel in which a plurality of light-transmitting attachment members are irregularly arranged, wherein a plurality of cells are attached to and cultured on the plurality of light-transmitting attachment members, photographing, by an image photographing device that is movable within the housing, the plurality of cells attached to and cultured on the plurality of light-transmitting attachment members irregularly arranged in the light-transmitting culture vessel, and irradiating light to the plurality of cells attached to and cultured on the plurality of light-transmitting attachment members irregularly arranged in the light-transmitting culture vessel, to allow the image photographing device to photograph each of the plurality of cells attached to and cultured on the plurality of light-transmitting attachment members irregularly arranged in the light-transmitting culture vessel, to be recognizable.

### Advantageous Effects of Disclosure

It is an effect of the present disclosure to provide an apparatus for observing cells comprising a movable image photographing device and a method of observing cells using the same, in which not only an image of cells is obtained when a device for obtaining an image of cells is in a stopped state, but also an image of cells cultured in an area where an image of cells is difficult or impossible to obtain is obtained when the device for obtaining an image of cells is in a stopped state, thereby obtaining an image of cells cultured in all areas, not in a limited area, so that the observation efficiency may be greatly improved.

It is another effect of the present disclosure to provide an apparatus for observing cells comprising a movable image photographing device and a method of observing cells using the same, in which not only an image of cells cultured in one culture vessel is obtained, but also an image of cells cultured in a plurality of culture vessels is obtained, thereby greatly improving the observation efficiency of cells cultured in a plurality of culture vessels.

It is another effect of the present disclosure to provide an apparatus for observing cells comprising a movable image photographing device and a method of observing cells using the same, in which an image of cells cultured in a culture medium in a culture vessel is easily obtained, thereby greatly improving the observation efficiency of cells cultured in a culture medium in a culture vessel.

It is another effect of the present disclosure to provide an apparatus for observing cells comprising a movable image photographing device and a method of observing cells using the same, in which a device for obtaining an image of cells is remotely driven as necessary, thereby greatly improving driving efficiency.

The effects of the present disclosure are not limited to the above-described effects, and other various effects that are not described in the present disclosure may be clearly understood from the following descriptions by one skilled in the art to which the present disclosure belongs.

### BRIEF DESCRIPTION OF DRAWINGS

FIG. 1 is a front cross-sectional view schematically illustrating an apparatus for observing cells comprising a movable image photographing device, according to an embodiment of the present disclosure.

FIG. 2 is a side cross-sectional view schematically illustrating an apparatus for observing cells comprising a movable image photographing device, according to an embodiment of the present disclosure.

FIG. 3 is a view illustrating a culture vessel photographed by an apparatus for observing cells comprising a movable image photographing device, according to an embodiment of the present disclosure.

FIG. 4 is a view schematically illustrating an apparatus for observing cells comprising a movable image photographing device, according to an embodiment of the present disclosure, the apparatus being connected to a computer device or a server.

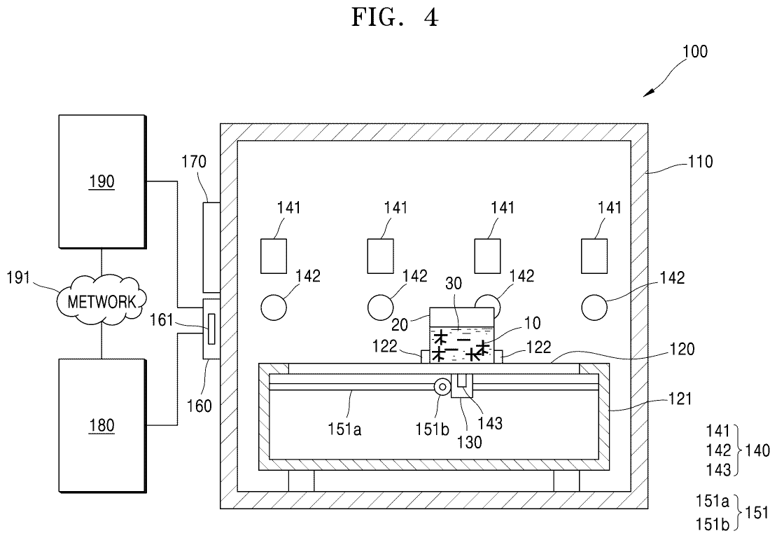

FIG. 5 is a flowchart schematically showing a method of observing cells comprising a movable image photographing device, according to another embodiment of the present disclosure.

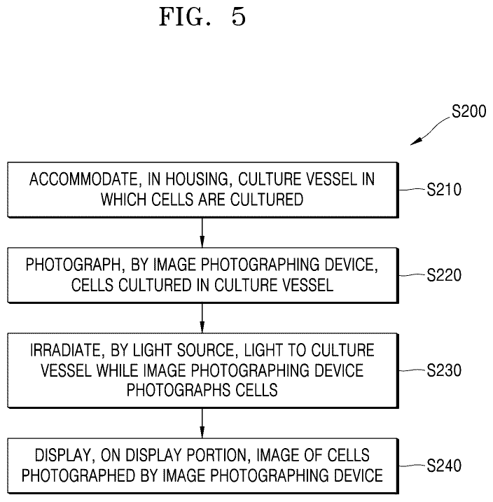

### MODE OF DISCLOSURE

Detailed descriptions to implement the disclosure are presented based on the embodiments. However, the present disclosure is not limited thereto and it will be understood that various changes in form and details may be made therein without departing from the spirit and scope of the following claims. That is, descriptions on particular structures or functions may be presented merely for explaining embodiments of the disclosure.

Furthermore, the terms used in the disclosure are merely used to describe embodiments, and are not intended to limit the disclosure. As used herein, the singular forms “a,” “an,” and “the” are intended to include the plural forms as well, unless the context clearly indicates otherwise.

In the specification, it will be further understood that terms “comprises” and/or “comprising” when used in this specification, specify the presence of stated features, numbers, steps, actions, components, parts, or combinations thereof, and are not intended to preclude the possibility that one or more other features, numbers, steps, actions, components, parts, or combinations thereof may exist or may be added.

### Embodiment 1

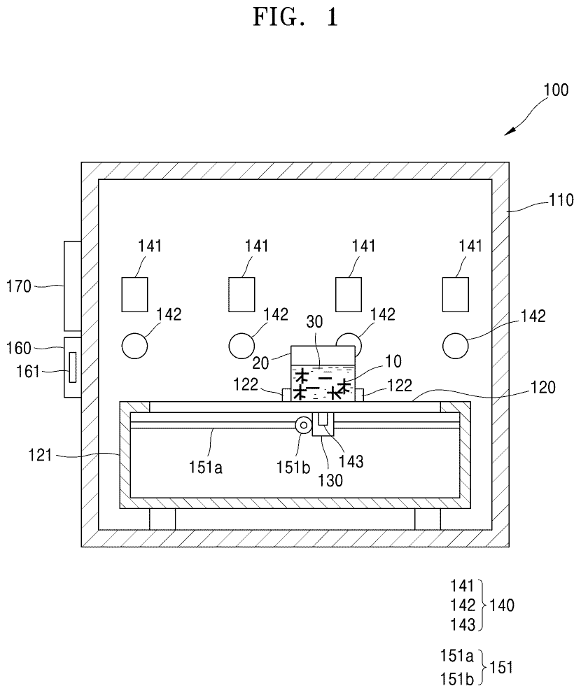

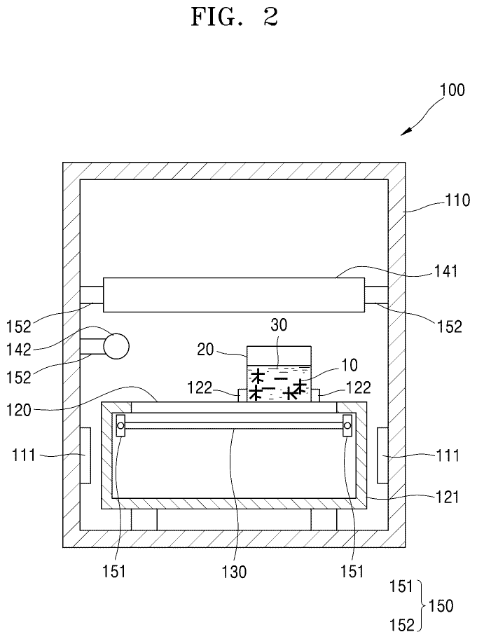

An apparatus 100 for observing cells comprising a movable image photographing device, according to an embodiment of the present disclosure, as illustrated in FIGS. 1 and 2 as an example, includes a housing 110, a support body 120, an image photographing device 130, a light source 140, and a driving portion 150. In addition, the apparatus 100 for observing cells comprising a movable image photographing device may further include a controller 160, a display portion 170, a computer device 180, and a server 190.

The housing 110 accommodates a culture vessel 20 in which cell 10 are cultured.

The cell 10 may include multifunctional stem cells, such as induced pluripotent stem (iPS) cells and embryonic stem (ES) cells, cells of nerve, skin, heart muscle, and liver induced to differentiate from stem cells, cells of skin, retina, heart muscle, hemocyte, nerve, and organ taken from the human body, and the like. Furthermore, the cell 10 may include not only one cell, but a cell group (cell colony) made up of a plurality of cells.

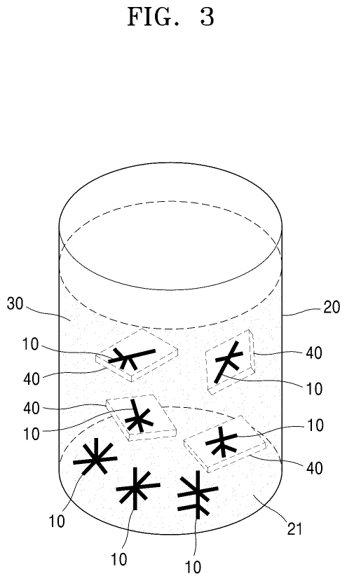

The culture vessel 20, as illustrated in FIG. 3 as an example, is where the cell 10 is accommodated and cultured and may include ingredients to culture the cell 10.

For example, the culture vessel 20 may include a culture medium 30 needed for cell culture. The culture medium 30 may be filled to a height at which the cell 10 is submerged so that the cell 10 may be appropriately cultured, for example, a height of 5 mm or more from a bottom 21 of the culture vessel 20.

In general, the cell 10 in the culture vessel 20 grows vertically by being attached to the bottom 21 that is flat due to the nature.

The culture vessel 20 may include a plurality of attachment members 40 to which the cell 10 is attached and cultured. Accordingly, the cell 10 may be smoothly cultured as the cell 10 may be cultured by being attached to the attachment members 40 in addition to the bottom 21 of the culture vessel 20.

The attachment members 40 may include various materials that enable attachment and culture of the cell 10. For example, the attachment members 40 may include a light-transmitting material such as a transparent film. The light-transmitting attachment members 40, when an image of the cell 10 attached thereto is photographed, may enable the image photographing device 130 to photograph an image of the cell 10 through the attachment members 40.

In addition, the housing 110 may further include a temperature control device that maintains a temperature within a range in which culture of the cell 10 is possible, for example, a temperature of 22° C. to 43° C. Such a temperature control device may include a device such as a heater 111 installed inside the housing 110.

The support body 120 is arranged to support the culture vessel 20 inside the housing 110. Accordingly, as the culture vessel 20 is arranged at a specific position inside the housing 110, an image may be obtained by photographing the cell 10 cultured in the culture vessel 20.

The support body 120 may have various configurations arranged to support the culture vessel 20 inside the housing 110.

For example, the support body 120, as illustrated in FIGS. 1 and 2 as an example, may have a plate shape of supporting the culture vessel 20 on an upper side thereof. The support body 120 of a plate shape may extend flat along a bottom surface in the housing 110. The support body 120 of a plate shape may be supported by a support 121 on the bottom of the housing 110.

The support body 120, as illustrated in FIGS. 1 and 2 as an example, may support the one culture vessel 20. However, the present disclosure is not limited thereto, and the support body 120 may support a plurality of culture vessels. Accordingly, by arranging the plurality of culture vessels 20 at specific positions inside the housing 110, an image of the cells 10 cultured in the plurality of culture vessels 20 may be photographed and obtained.

The support body 120, as illustrated in FIGS. 1 and 2 as an example, may include an auxiliary support 122 that assists the culture vessel 20 to be arranged at a specific position. Accordingly, by easily and accurately arranging the culture vessel 20 at a specific position inside the housing 110, the cell 10 cultured in the culture vessel 20 may be easily and accurately photographed. The auxiliary support 122, as illustrated in FIGS. 1 and 2 as an example, may be formed in a shape of a plurality of protrusions that protrude from an upper surface of the support body 120 to be in contact with the outer surface of the culture vessel 20 that is specific. However, the present disclosure is not limited thereto, and the auxiliary support 122 may have various other configurations to assist to arrange the culture vessel 20 at a specific position on the support body 120.

The support body 120 may include various materials to support the culture vessel 20.

For example, the support body 120 may include a light-transmitting material such as a transparent film. When the cell 10 in the culture vessel 20 supported thereon is photographed, the support body 120 may enable the cell 10 in the culture vessel 20 to be photographed through the support body 120 so that an image of the cell 10 in the culture vessel 20 supported on the support body 120 may be obtained.

However, the support body 120 may include a non-light-transmitting material such as an opaque film. The support body 120 that is opaque may support the culture vessel 20 and enable photographing the cell 10 in the culture vessel 20 by exposing the culture vessel 20 or a portion therein to which the cell 10 is attached without being covered by the support body 120 that is opaque. For example, by cutting off a portion of the support body 120 corresponding to an area of the bottom 21 of the culture vessel 20, and photographing the bottom 21 of the culture vessel 20 through the cut-off portion, the cell 10 that is attached to the bottom 21 of the culture vessel 20 and grows thereon or the cell 10 that is attached to the attachment members 40 in the culture vessel 20 and grows thereon may be photographed.

The image photographing device 130 is a device to photograph the cell 10 cultured in the culture vessel 20 while stopping or moving, as necessary, within the housing 110. Accordingly, as the image photographing device 130 may obtain not only an image of the cell 10 in a stopped state, but also an image of the cell 10 cultured in an area where the image photographing device 130 in a stopped state is difficult or impossible to obtain an image of the cell 10, the observation efficiency of the cell 10 cultured in all areas, not in a limited area, may be greatly improved.

The image photographing device 130 may be variously arranged to photograph the cell 10 cultured in the culture vessel 20.

For example, the image photographing device 130, as illustrated in FIGS. 1 and 2 as an example, may be arranged under the support body 120. The image photographing device 130 may linearly extend from one end portion of the support body 120 to the other end portion thereof, for example, a front end portion of the support body 120 to a rear end portion thereof. While moving along the lower surface of the support body 120, for example, from a left end portion of the support body 120 to a right end portion thereof or inversely, the image photographing device 130 arranged as above may photograph the cell 10 cultured in a single or a plurality of culture vessels 120 supported on the upper surface of the support body 120. In this state, in the image photographing device 130, one moving along the lower surface of the support body 120 under the support body 120 may be an optical system such as a lens or mirror that forms an image of the cell 10 in the culture vessel 120, whereas the other part, such as an imaging system such as an image sensor, and the like, may not move.

While stopping or moving, as necessary, within the housing 110, the image photographing device 130 may variously photograph the cell 10 cultured in the culture vessel 20. Accordingly, a specific area, such as an area where the cell 10 is cultured, may be easily and conveniently specified, and a high-resolution image of the cell 10 cultured in the specific area may be easily and conveniently generated. For example, the image photographing device 130 may primarily generate a first image by photographing the entire area to be photographed, such as one culture vessel 120 or a plurality of culture vessels 120, at a low resolution, and secondarily generate a second image by photographing a specific area such as a culture area corresponding to a specific portion such as a portion of the first image, where the cell 10 is cultured, at a high resolution.

The generation of the first image may be quickly performed by photographing the entire area to be photographed by the image photographing device 130 while moving in a series of sequences, and the generation of the second image may be performed as the image photographing device 130 clearly performs photographing at a high resolution through focusing and the like, in a stopped state, after having moved to a specific area such as an area where the cell 10 is cultured. The first image may be obtained, as a preview image, by photographing the entire area to select a specific area, and the second image may be one or a plurality of images obtained, as a cell observation image, by photographing one or a plurality of specific areas where the cell 10 is generated.

As the image photographing device 130 obtains not only an image of the cell 10 cultured in the one culture vessel 20, but also an image of the cells 10 cultured in the plurality of culture vessels 20, the observation efficiency of the cells 10 cultured in the plurality of culture vessels 20 may be greatly improved.

When the image photographing device 130 photographs the cell 10 cultured in the one culture vessel 20, the image photographing device 130 may obtain an image of the cell 10 by photographing the cell 10 in a stopped state at a position corresponding to the position of the culture vessel 20.

When the image photographing device 130 photographs the cells 10 cultured in the plurality of culture vessels 20, the image photographing device 130 may easily and conveniently obtain an image of the cell 10 by photographing the cell 10 cultured in each culture vessel 20 while moving in a series of sequences in areas corresponding to the positions of the plurality of culture vessels 20, for example, from an area corresponding to the position of the first culture vessel 20 to an area corresponding to the position of the last culture vessel 20.

The light source 140 irradiates light to the culture vessel 20, while the image photographing device 130 photographs the cell 10. Accordingly, when photographing the cell 10 of the culture vessel 20, the image photographing device 130 may easily and accurately photograph the cell 10 so that a high-resolution image of the cell 10 may be obtained. Accordingly, as the light source 140 easily and accurately obtains an image of the cell 10 cultured in the culture medium 30 in the culture vessel 20, the observation efficiency of the cell 10 cultured in the culture medium 30 in the culture vessel 20 may be greatly improved.

While stopping or moving, as necessary, within the housing 110, the light source 140 may irradiate light to the culture vessel 20. Accordingly, while stopping or moving, as necessary, within the housing 110, the light source 140 irradiates light to the culture vessel 20 corresponding to the image photographing device 130 photographing the cell 10 cultured in the culture vessel 20 while stopping or moving, as necessary, within the housing 110, and thus, light efficiency may be improved.

The light source 140 may include various light sources that irradiate light to the culture vessel 20. For example, the light source 140, as illustrated in FIGS. 1 and 2 as an example, may include a first light source 141, a second light source 142, and a third light source 143.

The first light source 141 irradiates light to the culture vessel 20 from above the culture vessel 20. Accordingly, the light irradiated from the first light source 141 passes downward the culture medium 30 that is turbid and red and filled in the culture vessel 20 to a certain depth, for example, a depth of 5 mm or more, and reaches the cell 10 that is attached to the bottom 21 of the culture vessel 20 and grows thereon or the cell 10 that is attached to the attachment members 40 suspended or precipitated in the culture medium 30 and grows thereon, thereby allowing the cell 10 to be clearly photographed.

The first light source 141 may have various configurations to irradiate light to the culture vessel 20 from above the culture vessel 20. For example, the first light source 141, as illustrated in FIGS. 1 and 2 as an example, may include a plurality of linear light sources that are arranged at positions higher than the height of the culture vessel 20 from the support body 120 supporting the culture vessel 20 and linearly extend from the front end portion of the support body 120 to the rear end portion thereof. However, the present disclosure is not limited thereto, and the first light source 141 may be a surface light source having a light-emitting surface corresponding to the upper surface of the support body 120 or a plurality of spot light sources arranged at positions corresponding to the plurality of culture vessels 20 located on the upper surface of the support body 120.

The first light source 141 may irradiate light in various methods. For example, the first light source 141 may irradiate light in conjunction with the image photographing device 130 photographing the cell 10 cultured in the culture vessel 20 while stopping or moving, as necessary, within the housing 110. Accordingly, the first light source 141 may improve light efficiency. When the image photographing device 130 in a stopped state within the housing 110 photographs the cell 10 cultured in the culture vessel 20 that is specific, the first light source 141 in a stopped state within the housing 110 may irradiate light to the culture vessel 20 only. When the image photographing device 130 moves within the housing 110 and photographs the cell 10 cultured in the culture vessel 20, the first light source 141 may irradiate light to the culture vessel 20 while moving within the housing 110.

The first light source 141 may irradiate light in various forms. For example, the first light source 141 may have a shape corresponding to the image photographing device 130, for example, a linear shape corresponding to the linear shape of the image photographing device 130, so as to irradiate light while stopping or moving in conduction with the image photographing device 130 photographing while stopping or moving as necessary.

The first light source 141 may irradiate light at various angles. For example, the first light source 141 may irradiate light at an angle of 90 degrees or less.

The second light source 142 irradiates light to the culture vessel 20 from the side portion of the culture vessel 20. Accordingly, the light irradiated from the second light source 142 passes through the culture medium 30 that is turbid and red and filled in the culture vessel 20 to a certain width, in a horizontal direction, and reaches the cell 10 that is attached to the bottom 21 of the culture vessel 20 and grows thereon or the cell 10 that is attached to the attachment members 40 suspended or precipitated in the culture medium 30 and grows thereon, thereby allowing the cell 10 to be clearly photographed.

The second light source 142 may have various configurations to irradiate light to the culture vessel 20 from the side portion of the culture vessel 20. For example, the second light source 142, as illustrated in FIGS. 1 and 2 as an example, may include a plurality of spot light sources that are arranged in a row from the left end portion of the support body 120 to the right end portion thereof along the rear end portion of the support body 120, at a height corresponding to the height of the cell 10 cultured in the culture vessel 20 supported by the support body 120. However, the present disclosure is not limited thereto, and the second light source 142 may be a linear light source that extends from the left end portion of the support body 120 to the right end portion thereof along the rear end portion of the support body 120 at a height corresponding to the height of the cell 10 cultured in the culture vessel 20 or further entirely surround the support body 120, or a surface light source in which the height of such a linear light source extends to correspond to the height of the culture vessel 20.

The second light source 142 may irradiate light in various methods. For example, the second light source 142 may irradiate light in conjunction with the image photographing device 130 photographing an image of the cell 10 cultured in the culture vessel 20 while stopping or moving, as necessary, within the housing 110. Accordingly, the second light source 142 may improve light efficiency. When the image photographing device 130 in a stopped state within inside the housing 110 photographs an image of the cell 10 cultured in the culture vessel 20 that is specific, the second light source 142 may be controlled to irradiate light to the culture vessel 20 only. When the image photographing device 130 moves inside the housing 110 and photographs an image of the cell 10 cultured in the culture vessel 20, the second light source 142 may be controlled to irradiate light in conjunction with the movement of the image photographing device 130.

The second light source 142 may irradiate light in various methods. For example, the second light source 142 may irradiate light in conjunction with the image photographing device 130 photographing the cell 10 cultured in the culture vessel 20 while stopping or moving, as necessary, within the housing 110. Accordingly, the second light source 142 may improve light efficiency. When the image photographing device 130 in a stopped state within the housing 110 photographs the cell 10 cultured in the culture vessel 20 that is specific, the second light source 142 in a stopped state within inside the housing 110 may irradiate light to the culture vessel 20 only. When the image photographing device 130 moves within the housing 110 and photographs the cell 10 cultured in the culture vessel 20, the second light source 142 may irradiate light to the culture vessel 20 while moving within the housing 110.

The second light source 142 may irradiate light at various angles. For example, the second light source 142 may irradiate light at an angle of 90 degrees or less.

The third light source 143 irradiates light to the culture vessel 20 from under the culture vessel 20. Accordingly, the light irradiated from the third light source 143 passes upward through the bottom 21 of the culture vessel 20 and reaches the cell 10 that is attached to the bottom 21 and grows thereon or the cell 10 that is attached to the attachment members 40 suspended or precipitated in the culture medium 30 and grows thereon, thereby allowing the cell 10 to be clearly photographed.

The third light source 143 may have various configurations to irradiate light to the culture vessel 20 from under the culture vessel 20. For example, the third light source 143, as illustrated in FIGS. 1 and 2 as an example, may be arranged under the support body 120 supporting the culture vessel 20 and may include a plurality of linear light sources linearly extending from the front end portion of the support body 120 to the rear end portion thereof. However, the present disclosure is not limited thereto, and the third light source 143 may be a surface light source having a light-emitting surface corresponding to the lower surface of the support body 120 or a plurality of spot light sources arranged at positions corresponding to the plurality of culture vessels 20 located on the support body 120.

The third light source 143 may irradiate light in various methods. For example, the third light source 143 may irradiate light in conjunction with the image photographing device 130 photographing the cell 10 cultured in the culture vessel 20 while stopping or moving, as necessary, within the housing 110. Accordingly, the third light source 143 may improve light efficiency. When the image photographing device 130 in a stopped state within the housing 110 photographs the cell 10 cultured in the culture vessel 20 that is specific, the third light source 143 in a stopped state within inside the housing 110 may irradiate light to the culture vessel 20 only. When the image photographing device 130 moves within the housing 110 and photographs the cell 10 cultured in the culture vessel 20, the third light source 143 may irradiate light to the culture vessel 20 while moving within the housing 110.

The third light source 143 may irradiate light at various angles. For example, the third light source 143 may irradiate light at an angle of 90 degrees or less.

The third light source 143 may stop or move, as necessary, with the image photographing device 130. Accordingly, only when the image photographing device 130 while stopping or moving, as necessary, within the housing 110 photographs the cell 10 cultured in the culture vessel 20, the light from the third light source 143 may be irradiated to the cell 10, and thus, a high-resolution cell image may be easily and conveniently obtained and the observation efficiency may be greatly improved.

The third light source 143 may have various configurations to move with the image photographing device 130. For example, the third light source 143 may stop or move, as necessary, with the image photographing device 130 by being coupled to the image photographing device 130. In this state, the third light source 143 may be coupled to a side surface of the image photographing device 130 or installed inside the image photographing device 130 to be arranged on an upper surface of the image photographing device 130 facing the culture vessel 20.

The driving portion 150 drives the image photographing device 130 to stop or move, as necessary, within the housing 110 or drives the light source 140 to stop or move, as necessary, within the housing 110. Accordingly, an image of the cell 10 cultured in the culture vessel 20 inside the housing 110 is easily and conveniently obtained by stopping or moving, as necessary, the image photographing device 130 or the light source 140, and thus the observation efficiency may be greatly improved.

The driving portion 150 may move the image photographing device 130 to a specific position to be photographed, while the image photographing device 130 is in a stopped state, or move the image photographing device 130 to allow the image photographing device 130 to photograph while moving.

Furthermore, the driving portion 150 may move the light source 140 to a specific position to be photographed, while the image photographing device 130 is in a stopped state, or move the light source 140 to allow the image photographing device 130 to photograph while moving.

The driving portion 150 may include a first driving portion 151 and a second driving portion 152 according to an object to move.

The first driving portion 151 may move the image photographing device 130.

The first driving portion 151 may have various configurations to move the image photographing device 130. For example, the first driving portion 151 may include a guide shaft 151a that guides the reciprocation of the image photographing device 130 and a motor 151b that drives the reciprocation of the image photographing device 130 through the guide of the guide shaft 151a.

The second driving portion 152 moves the light source 140.

The second driving portion 152 may have various configurations to move the light source 140. For example, the second driving portion 152 may include a guide shaft (not shown) that guides the reciprocation of the light source 140 and a motor (not shown) that drives the reciprocation of the light source 140 according to the guide of the guide shaft.

The controller 160 controls the image photographing device 130, the light source 140, and the driving portion 150. For example, the controller 160 may control the image photographing device 130 to photograph the cell 10 cultured in the culture vessel 20 while stopping or moving, as necessary, within the housing 110. The controller 160 may control the light source 140 to irradiate light to the culture vessel 20 while stopping or moving, as necessary, within the housing 110. The controller 160 may control the driving portion 150 to drive the image photographing device 130 to stop or move, as necessary, within the housing 110. The controller 160 may control the driving portion 150 to drive the light source 140 to stop or move, as necessary, within the housing 110.

The controller 160 may have various configurations to control the image photographing device 130, the light source 140, and the driving portion 150. For example, the controller 160 may be configured to include a processor 161 to control the image photographing device 130, the light source 140, and the driving portion 150.

The controller 160 may be arranged at various positions. For example, the controller 160, as illustrated in FIG. 1 as an example, may be arranged outside the housing 110. Accordingly, the controller 160 may control, outside the housing 110, the image photographing device 130, the light source 140, and the driving portion 150. In this case, the controller 160 may be electrically connected by a cable 162 to the image photographing device 130, the light source 140, and the driving portion 150 inside the housing 110. In this state, the controller 160 may be installed on an outer surface of the housing 110.

The display portion 170 displays an image of the cell 10 generated through the photographing of the image photographing device 130. Accordingly, an observer may easily and conveniently observe an image of the cell 10.

Meanwhile, the apparatus 100 for observing cells comprising a movable image photographing device may be electrically connected to the computer device 180 that is externally arranged, through wired or wireless communication. Accordingly, the observer may easily and conveniently observe an image of the cell 10 in the apparatus 100 for observing cells through the external computer device 180. Here, the computer device 180 may include various devices that are electrically connected to the apparatus 100 for observing cells, such as a desktop personal computer (PC), a smartphone, a notebook computer, a tablet PC, and the like, through wired or wireless communication. Here, as the computer device 180 is electrically connected to the controller 160 of the apparatus 100 for observing cells, the computer device 180 may control, through the processor 161, the image photographing device 130, the light source 140, and the driving portion 150. Accordingly, the image photographing device 130, the light source 140, and the driving portion 150 may be controlled outside the apparatus 100 for observing cells.

Furthermore, the apparatus 100 for observing cells comprising a movable image photographing device may be directly connected, via an Internet network 191, to the server 190 that is externally arranged or indirectly connected to the server 190 that is externally arranged, via the Internet network 191 through the computer device 180. Accordingly, by the server 190 that is externally arranged and directly connected via the Internet network 191 or indirectly connected via the Internet network 191, through the computer device 180, the cell 10 inside the apparatus 100 for observing cells may be easily and conveniently observed. Accordingly, as a device for obtaining an image of cells is remotely driven as necessary, driving efficiency may be greatly improved.

Here, the server 190 may be connected to a plurality of apparatuses 100 for observing cells or a plurality of computer devices 180 so that the cell 10 from the plurality of apparatuses 100 for observing cells or the plurality of computer devices 180 may be easily and conveniently observed. The server 190 may manage the culture state of the cell 10 by receiving an image of the cell 10 from the plurality of apparatuses 100 for observing cells or the plurality of computer devices 180. The server 190 may receive and store the image of the cell 10 photographed by the image photographing device 130 from the plurality of apparatuses 100 for observing cells or the plurality of computer devices 180.

Meanwhile, although not illustrated in the drawings, the apparatus 100 for observing cells according to the present embodiment may further include a reflection portion that reflects the light irradiated from the light source 140 toward a plurality of cells 10 attached to and cultured on the attachment members 40 that are light-transmitting and irregularly arranged in the culture vessel 20. Such a reflection portion reflects the light irradiated from the light source 140 to be irradiated to the plurality of cells 10 in culture so that the plurality of cells 10 in culture are more irradiated with light. Accordingly, the plurality of cells 10 in culture may be more clearly observed so that observation efficiency may be further improved.

The reflection portion may be variously configured to reflect the light irradiated from the light source 140 toward the plurality of cells 10 attached to and cultured on the attachment members 40 that are light-transmitting and irregularly arranged in the culture vessel 20.

For example, the reflection portion may be located within an irradiation angle range of the light source 140 and may reflect the light irradiated from the light source 140 toward the plurality of cells 10 attached to and cultured on the attachment members 40 that are light-transmitting and irregularly arranged in the culture vessel 20.

Furthermore, the reflection portion may be located opposite the light source 140 and may reflect the light irradiated from the light source 140 toward the plurality of cells 10 attached to and cultured on the attachment members 40 that are light-transmitting and irregularly arranged in the culture vessel 20.

### Embodiment 2

A method of observing cells comprising a movable image photographing device according to another embodiment of the present disclosure may use the apparatus 100 for observing cells of the embodiment described above.

The method of observing cells comprising a movable image photographing device according to the present embodiment (S200) includes accommodating, in a housing, a light-transmitting culture vessel in which cells are cultured (S210). The culture vessel may be accommodated in the housing in a state of being filled to a certain height with a culture medium to culture cells therein to submerge the cells. The culture vessel may be accommodated to be arranged on a support within the housing. The housing may maintain a temperature within a range in which cell culture is possible.

The cells cultured in the culture vessel accommodated in the housing are photographed by an image photographing device (S220). In this state, while stopping or moving, as necessary, within the housing may photograph, the image photographing device may photograph the cells cultured in the culture vessel. For example, while moving under the support along a lower surface of the support, the image photographing device may photograph the cells cultured in a plurality of culture vessels supported on an upper surface of the support. In this state, while moving along the entire area in which one culture vessel or a plurality of culture vessels are arranged, the image photographing device may perform preview photographing of generating a first image by primarily photographing the entire area. Then, the image photographing device may repeat moving and stopping across specific areas corresponding to a specific portion in the first image, for example, areas in which cells are cultured or specific culture vessels, in a series of sequences, and then, perform high-resolution photographing of generating second images by secondarily photographing a specific area at a resolution higher than the resolution of the first image through focusing and the like.

Driving the image photographing device to stop or move, as necessary, within housing may be performed by a separate driving portion.

While the image photographing device photographs cells, the light source may irradiate light to the culture vessel (S230). The light source may irradiate light to the culture vessel while stopping or moving, as necessary, within the housing. For example, while stopping or moving, as necessary, within the housing, the light source may irradiate light to the culture vessel in conjunction with the image photographing device stopping or moving, as necessary, within housing. When the image photographing device moves along the entire area in which one culture vessel or a plurality of culture vessels are arranged, and performs preview photographing of generating a first image by primarily photographing the entire area, the light source may irradiate light to the entire area while moving along the entire area in which one culture vessel or a plurality of culture vessels are arranged. Then, when the image photographing device repeats moving and stopping across specific areas corresponding to a specific portion in the first image, for example, areas in which cells are cultured, in a series of sequences, and then performs high-resolution photographing of generating second images by secondarily photographing a specific area at a resolution higher than the resolution of the first image through focusing and the like, the light source may repeat moving and stopping across the specific areas, such as the areas in which cells are cultured, in a series of sequences, and irradiate light to the specific areas.

The irradiation of light to the culture vessel may include first irradiation of irradiating light to the culture vessel from above the culture vessel, second irradiation of irradiating light to the culture vessel from the side of the culture vessel, and third irradiation of irradiating light to the culture vessel from under the culture vessel. The third irradiation may be made by the light source that moves with the image photographing device. Driving the light source to stop or move, as necessary, within housing may be performed by a separate driving portion.

In addition, the driving of the image photographing device, the light source, and the driving portion may be controlled by a separate controller. Furthermore, the driving of the image photographing device, the light source, and the driving portion may be controlled by a separate computer device or an external server connected via the Internet network.

The cell image photographed by the image photographing device may be displayed on a display portion (S240). Furthermore, the cell image photographed by the image photographing device may be displayed on a display portion of a separate computer device or a display portion of an external server connected via the Internet network.

### INDUSTRIAL APPLICABILITY

The present disclosure may be used for an apparatus for observing cells and a method of observing cells using the same.

1. An apparatus for observing cells comprising a movable image photographing device, the apparatus comprising:
a housing accommodating a light-transmitting culture vessel in which a plurality of light-transmitting attachment members are irregularly arranged, wherein a plurality of cells are attached to and cultured on the plurality of light-transmitting attachment members;
an image photographing device movable within the housing and configured to photograph the plurality of cells attached to and cultured on the plurality of light-transmitting attachment members irregularly arranged in the light-transmitting culture vessel; and
a light source irradiating light to the plurality of cells attached to and cultured on the plurality of light-transmitting attachment members irregularly arranged in the light-transmitting culture vessel, to allow the image photographing device to photograph each of the plurality of cells attached to and cultured on the plurality of light-transmitting attachment members irregularly arranged in the light-transmitting culture vessel, to be recognizable.

2. The apparatus of claim 1, wherein the culture vessel is filled with a culture medium to culture cells to a certain height to submerge the plurality of cells.

3. The apparatus of claim 1, wherein the housing maintains a temperature within a range in which cell culture is possible.

4. The apparatus of claim 1, wherein the image photographing device is further configured to photograph, from under the culture vessel across a lower surface of the culture vessel, the plurality of cells attached to and cultured on the plurality of light-transmitting attachment members irregularly arranged in the culture vessel.

5. The apparatus of claim 1, wherein, while moving within the housing, the light source irradiates light to the plurality of cells attached to and cultured on the plurality of light-transmitting attachment members irregularly arranged in the culture vessel.

6. The apparatus of claim 1, wherein the light source comprises a first light source irradiating light, from above the culture vessel, to the plurality of cells attached to and cultured on the plurality of light-transmitting attachment members irregularly arranged in the culture vessel.

7. The apparatus of claim 1, wherein the light source comprises a second light source irradiating light, from a side portion of the culture vessel to the plurality of cells attached to and cultured on the plurality of light-transmitting attachment members irregularly arranged in the culture vessel.

8. The apparatus of claim 1, further comprising a reflection portion reflecting the light irradiated from the light source to the plurality of cells attached to and cultured on the plurality of light-transmitting attachment members irregularly arranged in the culture vessel.

9. The apparatus of claim 8, wherein the reflection portion is located within an irradiation angle range of the light source and reflects the light irradiated from the light source to the plurality of cells attached to and cultured on the plurality of light-transmitting attachment members irregularly arranged in the culture vessel.

10. The apparatus of claim 9, wherein the reflection portion is located opposite the light source and reflects the light irradiated from the light source to the plurality of cells attached to and cultured on the plurality of light-transmitting attachment members irregularly arranged in the culture vessel.

11. The apparatus of claim 1, further comprising a controller configured to control the image photographing device and the light source.

12. The apparatus of claim 11, wherein the controller is connected to an external server through wired/wireless communication, and the image photographing device or the light source is controlled by the external server.

13. The apparatus of claim 12, wherein the server receives an image of the plurality of cells photographed by the image photographing device.

14. The apparatus of claim 13, wherein the server determines a culture state of the plurality of cells by comparing information about the image of the plurality of cells photographed by the image photographing device with information about an image of the plurality of cells previously stored.

15. The apparatus of claim 1, further comprising a display portion displaying an image of the plurality of cells photographed by the image photographing device.

16. A method of observing cells using an apparatus for observing cells including a movable image photographing device, the method comprising:
accommodating, in a housing, a light-transmitting culture vessel in which a plurality of light-transmitting attachment members are irregularly arranged, wherein a plurality of cells are attached to and cultured on the plurality of light-transmitting attachment members;
photographing, by an image photographing device that is movable within the housing, the plurality of cells attached to and cultured on the plurality of light-transmitting attachment members irregularly arranged in the light-transmitting culture vessel; and
irradiating light to the plurality of cells attached to and cultured on the plurality of light-transmitting attachment members irregularly arranged in the light-transmitting culture vessel, to allow the image photographing device to photograph each of the plurality of cells attached to and cultured on the plurality of light-transmitting attachment members irregularly arranged in the light-transmitting culture vessel, to be recognizable.
